(19) United States
(12) Patent Application Publication
(10) Pub. No.: US 2019/0226099 A1
(43) Pub. Date: Jul. 25, 2019

(54) CONSTRUCTIVE ARRANGEMENT INTRODUCED IN AN OXIDIZING SOLUTION GENERATOR

(71) Applicant: Hidrogeron do Brasil Comércio de Equipamentos Para Saneamento Ambiental Ltda., Arapongas (BR)

(72) Inventor: Dionisius Duarte Rossetto RIBEIRO DOS SANTOS, Arapongas (BR)

(21) Appl. No.: 15/914,675

(22) Filed: Mar. 7, 2018

(30) Foreign Application Priority Data
Jan. 25, 2018 (BR) 20 2018 001652 6

(51) Int. Cl. C25B 9/06; C25B 15/08; C02F 1/467; C02F 1/461; C25B 1/26

(52) U.S. Cl. CPC C25B 9/06; C25B 15/08; C25B 1/26; C02F 1/46104; C02F 1/4674

(57) ABSTRACT

An oxidizing solution generator wherein all elements that make out the oxidizing solution generator are coupled, forming a novel arrangement. The structural arrangement comprises a parallelepipedal metallic structure, preferably aluminum, having two small reservoirs attached to its back side, one for containing water and one for brine, the reservoirs connected to water and brine dispensers positioned above them. A sodium hypochlorite or oxidizing solution dispenser is also provided parallel to the dispensers, and is connected to the sodium hypochlorite or oxidizing solution reservoir, positioned on the front side of the structure on a lower board. A voltage current source is also provided on the front side of the higher portion of the structure parallel to a control switchboard, and an electrolytic reactor is provided in the intermediate region. An embodiment for generators sized to produce over 6 kg/day is also described.

### FIELD OF THE INVENTION

The present invention is in the chemical field of oxidizing solution generators.

### BACKGROUND OF THE INVENTION

The current oxidizing solution generators of the prior art do not have a specific structural arrangement defined in a such way as to optimize their use, and also present problems in relation to the storage and transport thereof.

As an example of an oxidizing solution generator that forms the present state of the art is found in document PI1105839-0 (BR) of the same Applicant as that of the present application. This machine is related to an oxidizing solution generator used to carry out the electrochemical reaction of sodium chloride (kitchen salt), thus generating a bactericidal solution. The static generator of this machine is comprised of “a brine reservoir, a chlorine generator source, a chlorine generator reactor and a chlorine dispenser; said continuous generator having a reservoir being constituted by a brine reservoir, a brine dispenser, a chlorine generator source, a chlorine generator reactor, a chlorine reservoir and a chlorine dispenser; said continuous generator having a saturating means being constituted by a brine dispenser, a chlorine generator source, a chlorine generator reactor, a chlorine reservoir, a chlorine dispenser, a saturating means automation switchboard, a salt saturating means, a water reservoir and a water dispenser.” However, it is notable that there is not a defined structural arrangement of the elements of this generator, and so the parts are randomly located, which results in inconveniences in the transport, storage and control of the components of the generator.

What is needed therefore is a structural arrangement of an oxidizing solution generator, wherein the integral components of the generator are organized in a metallic structure that allows for the improved use, transportation, storage and control of its components, solving the above-noted problems as present in current models.

### SUMMARY OF THE INVENTION

The present Application provides a structural arrangement of an oxidizing solution generator, wherein the all of the elements and/or integral components of that make out the oxidizing solution generator are coupled/organized in a metallic structure that allows for the improved use, transportation, storage and control of its components, forming a novel structural arrangement through which advantageous practical, safe and functional results are attained.

The new structural arrangement of an oxidizing solution generator of the present Application offers multiple advantages in relation to similar products currently available or known: it provides customers with full autonomy, preventing the transportation of several units separately; it is practical to install, as the customer themselves can install the generator, saving expenses of specialized workmanship; and it is possible to store the generator in a more organized way.

### BRIEF DESCRIPTION OF THE DRAWINGS

FIG. 1 is a perspective view of the new structural arrangement of an oxidizing solution generator.

FIG. 2 is a front view of the oxidizing solution generator.

FIG. 3 is a back view of the oxidizing solution generator.

FIG. 4 is an upper view of the oxidizing solution generator.

FIG. 4 is a right side view of the oxidizing solution generator.

FIG. 6 is a left side view of the oxidizing solution generator.

FIG. 7 is a perspective view of a constructive embodiment of the oxidizing solution generator.

FIG. 8 is a front view of a constructive embodiment of the oxidizing solution generator.

FIG. 9 is an upper view of a constructive embodiment of the oxidizing solution generator.

FIG. 10 is a side view of a constructive embodiment of the oxidizing solution generator.

### DETAILED DESCRIPTION OF THE INVENTION

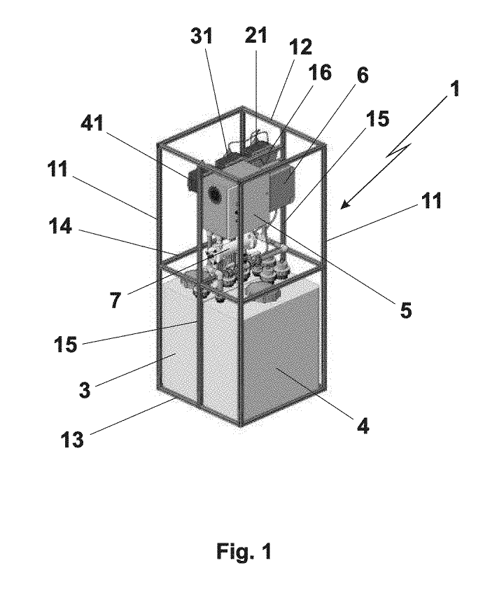

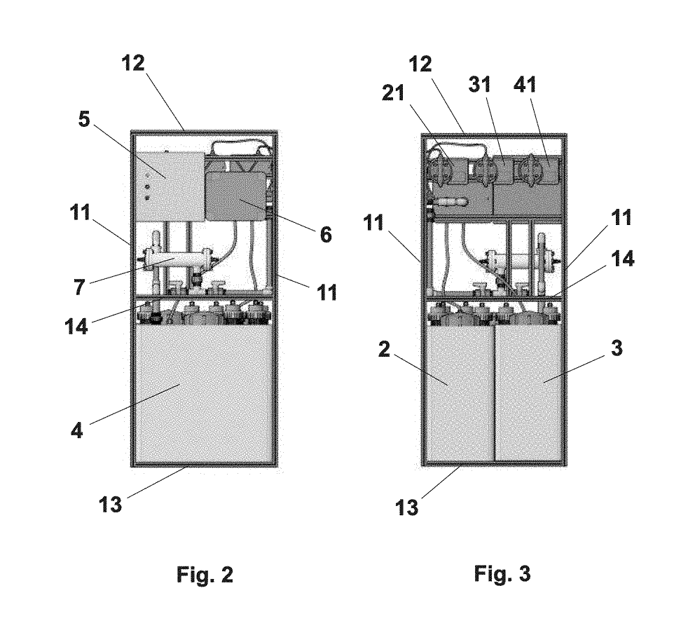

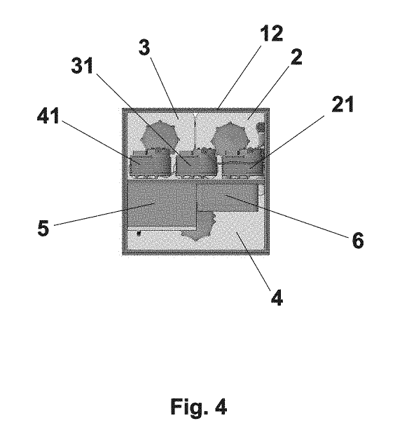

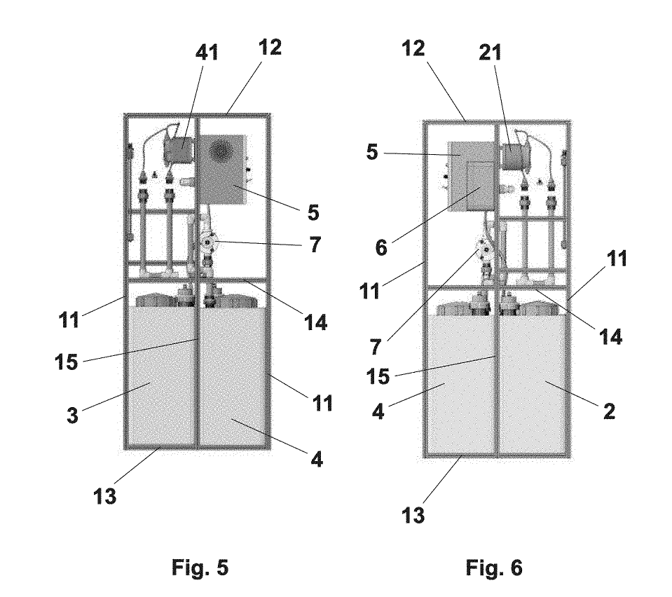

As shown in FIGS. 1 through 6, the new structural arrangement of an oxidizing solution generator comprises a parallelepipedal metallic structure (1), preferably made of aluminum, formed by four vertical corner rods (11), with dry fittings that join upper (12), lower (13) and intermediary boards (14), with two vertical side rods (15) also provided, the side rods joined at their upper portion by a transversal rod (16).

Inside the metallic structure (1) located in the back portion of the structure, two smaller reservoirs made of plastic (PP (polypropylene) or HDPE (high density polyethylene)) are coupled parallel to, above and upon the lower board (13), one reservoir for containing water (2) and the other one for containing brine (3). The reservoirs are connected to the water (21) and brine (31) dispensers, each dispenser being positioned above its respective reservoir. A sodium hypochlorite or oxidizing solution dispenser (41) is also provided parallel to the water and brine dispensers, the oxidizing solution dispenser connected to the sodium hypochlorite or oxidizing solution reservoir (4), with the oxidizing solution reservoir positioned in the front portion of the metallic structure above and upon the lower board (13).

The voltage current source (5), which is 110/220 or 380 VCA, is provided in the front portion of the upper portion of the structure, and is located parallel to a control switchboard (6) for the automation of solenoid valves that feed the reservoirs. An electrolytic reactor (7) is provided in the intermediate region of the structure, the reactor being comprised of anodes and cathodes made of titanium plates, wherein the brine solution is transformed into an oxidizing solution.

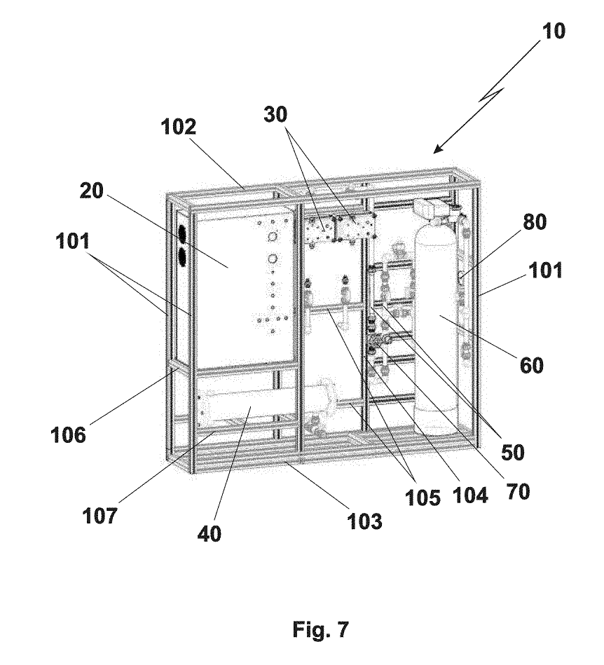

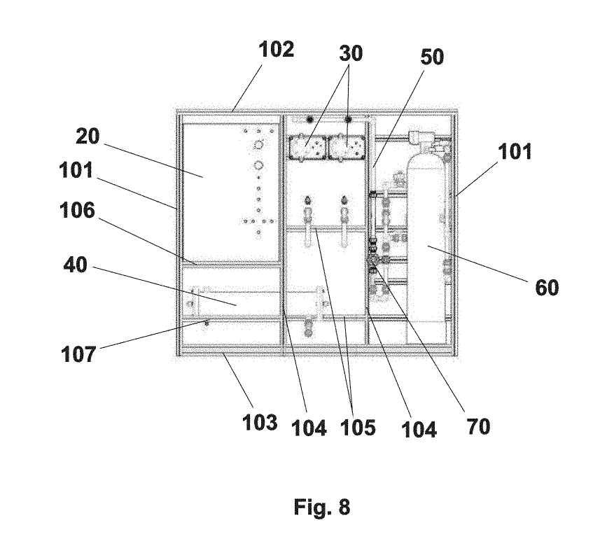

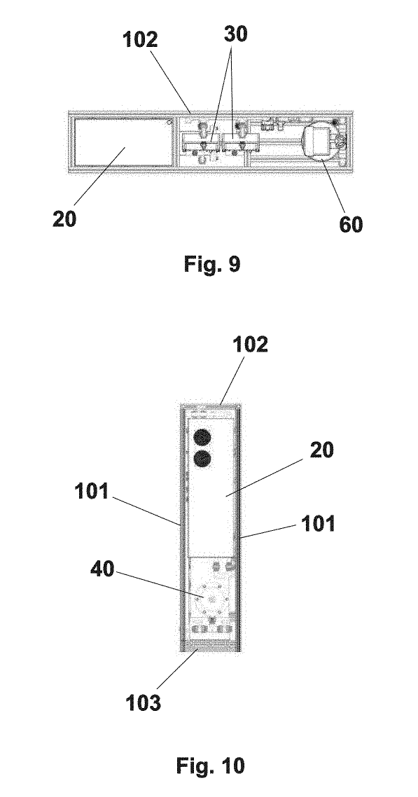

An embodiment of the present Application is show in FIGS. 7 through 10, useful for when the generator is sized to produce over 6 Kg/day of oxidizing solution. In this embodiment, the water, brine and sodium hypochlorite or oxidizing solution reservoirs are positioned outside the metallic structure. The structure is formed by a parallelepipedal metallic structure (10) comprising four vertical corner rods (101), with dry fittings that join the upper (102) and lower (103) boards, the structure additionally provided with two vertical back rods (104) joined by horizontal rods (105), with an upper intermediate board (106) also provided on one side of the intermediate portion of the structure.

In said metallic structure (10), the current source (20) is assembled on the upper intermediate board (106), parallel to the salt dispenser (30) located in the upper portion of the structure, and the electrolytic reactor (40) is coupled to a lower intermediate board (107) under the current source (20). The electrolytic reactor is connected by a pipe (50) to the softener (60), wherein the feeding water is controlled by a flow rate controller (70) and the pressurization is controlled by a pressure gauge (80).

In order to start the operation of the system using the new structural arrangement of an oxidizing solution generator of the present Application, the operator will require a water site, an electric power site and a site for applying the generated solution. The operation of the generator is continuously and electrically controlled (start and stop) through the control switchboard (6). When the sodium hypochlorite or oxidizing solution reservoir (4) is full, the generator is turned off automatically, and only the sodium hypochlorite or oxidizing solution dispenser (41) remains functioning. A microprocessor-controlled analyzer can be installed and connected to the sodium hypochlorite or oxidizing solution dispenser (41), for automatically adjusting the amount of product that the dispensers are injecting into the generator.

It should be understood that the preferred embodiments mentioned here are merely illustrative of the present invention. Numerous variations in design and use of the present invention may be contemplated in view of the following claims without straying from the intended scope and field of the invention herein disclosed.

1. An oxidizing solution generator, comprising:
(a) a parallelepiped-shaped metallic structure (1), having a front portion, a back portion, an upper portion, a lower portion, and an intermediate portion; the metallic structure formed by:
(a1) at least one upper board (12), at least one lower board (13) and at least one intermediary (14) board;
(a2) four vertical corner rods (11) the rods having dry fittings that join the at least one upper board (12), the at least one lower board (13) and the at least one intermediary (14) board; and
(a3) two vertical side rods (15), the vertical side rods joined at an upper portion by a transversal rod (16);
(b) a water reservoir (2) located within the lower back portion of the metallic structure, above and upon the at least one lower board (13);
(c) a brine reservoir (3) located within the lower back portion of the metallic structure, above and upon the at least one lower board (13); coupled parallel to the water reservoir;
(d) an oxidizing solution reservoir (4) positioned in the lower front portion of the metallic structure above and upon the lower board (13); coupled parallel to the water and brine reservoir;
(e) a water dispenser (21) located within the upper portion of the metallic structure, connected to and positioned above the water reservoir;
(f) a brine dispenser (31) located within the upper portion of the metallic structure, connected to and positioned above the brine reservoir;
(g) an oxidizing solution dispenser (41) located within the upper portion of the metallic structure; connected to and positioned above the oxidizing solution reservoir; and parallel to the water and brine dispensers;
(h) a voltage current source (5) located within the upper front portion of the metallic structure;
(i) a control switchboard (6), located within the upper front portion of the metallic structure; parallel to the voltage current source; and
(j) an electrolytic reactor (7) located within the intermediate region of the metallic structure, the electrolytic reactor comprising anodes and cathodes made of titanium plates.

2. The oxidizing solution generator of claim 1, wherein the voltage current source provides a voltage of 110/220 or 380 VCA.

3. The oxidizing solution generator of claim 1, wherein the metallic structure is of aluminum.

4. The oxidizing solution generator of claim 1, wherein the water reservoir and the brine reservoir are made of a plastic selected from the group consisting of: polypropylene and high density polyethylene.

5. An oxidizing solution generator, comprising:
(a) a parallelepiped-shaped metallic structure (10), having a front portion, a back portion, an upper portion, a lower portion, and an intermediate portion, the metallic structure formed by:
(a1) at least one upper board (102) and at least one lower board (103),
(a2) four vertical corner rods (101), with dry fittings that join the at least one upper board (102) and at the least one lower board (103),
(a3) two vertical back rods (104) on at least one side of the structure, the vertical back rods joined by horizontal rods (105), and
(a4) at least one upper intermediate board (106) located on a side of the intermediate portion of the structure,
wherein the water reservoir, the brine reservoir and the oxidizing solution reservoirs are positioned outside the metallic structure;
(b) a current source (20) located within the upper intermediate region of the metallic structure, the current source assembled above and located upon the upper intermediate board (106);
(c) a salt dispenser (30) located within the upper portion of the metallic structure and parallel to the current source; and
(d) an electrolytic reactor (40) located beneath the current source (20) within the lower portion of the metallic structure and coupled to a lower intermediate board (107); the electrolytic reactor connected by a pipe (50) to a softener (60), wherein the feeding of water is controlled by a flow rate controller (70) and the pressurization is controlled by a pressure gauge (80).

6. The oxidizing solution generator of claim 5, wherein the metallic structure is of aluminum.

7. The oxidizing solution generator of claim 5, wherein the water reservoir and the brine reservoir are made of a plastic selected from the group consisting of: polypropylene and high density polyethylene.
